(19) United States
(12) Patent Application Publication
(10) Pub. No.: US 2010/0324408 A1
(43) Pub. Date: Dec. 23, 2010

(54) IMPLANTABLE SYSTEM FOR DETERMINING THE ACCOMMODATION REQUIREMENT BY MEASURING THE EYEBALL ORIENTATION USING AN EXTERNAL MAGNETIC FIELD

(76) Inventors: Simon Klink, Stuttgart (DE); Georg Bretthauer, Karlsruhe (DE); Rudolf Guthoff, Rostock (DE); Ulrich Gengenbach, Remchingen (DE); Mark Bergemann, Goppingen (DE); Torsten Koker, Stutensee (DE); Wolfgang Rückert, Wulfrath (DE)

Correspondence Address: LUCAS & MERCANTI, LLP, 475 PARK AVENUE SOUTH, 15TH FLOOR, NEW YORK, NY, 10016, US

(21) Appl. No.: 12/449,617

(22) PCT Filed: Feb. 18, 2008
(86) PCT No.: PCT/EP2008/051938

§ 371 Date: Sep. 14, 2009

(30) Foreign Application Priority Data
Feb. 21, 2007 (DE) 10 2007 008 374.4

(51) Int. Cl. A61B 5/05

(52) U.S. Cl. 600/409

(57) ABSTRACT

The present invention relates to an implantable system for determining the accommodation requirement in an artificial accommodation system by measuring the eyeball orientation using an external magnetic field, comprising
a) at least one optical system (3),
b) at least one data acquisition system (8) which does not contact the ciliary muscle and has means for measuring a spatial orientation of both eyeballs as a physical control signal for the accommodation requirement,
c) at least one data processing system (9) for generating an actuating signal for the optical system (3) from the measured physical control signals,
d) at least one energy supply system (10), and
e) one fixing system,
   wherein the system in each case has means for measuring a magnetic field in both eyes and provision is made for transfer means for mutual information exchange between the means.

The present application claims the priority of 10 2007 008 374.4-55. The priority document is incorporated by reference in its entirety in the present disclosure.

The subject matter of the invention is an implantable system for determining the accommodation requirement in an artificial accommodation system by measuring the eyeball orientation using an external magnetic field.

The human eye is an optical system which uses a number of refractive interfaces to image focused objects on the retina. In the process, the light waves pass the cornea, the aqueous humor in the anterior chamber of the eye (camera anterior bulbi), the lens (lens crystallina) and the vitreous humor in the posterior segment of eyeball (camera vitrea bulbi), all of which have different refractive indices. If the object distance of the observed object changes, the imaging behavior of the optical system has to change in order to maintain an unchangingly focused image on the retina. The human eye implements this by deforming the lens using the ciliary muscle (musculus ciliaris); as a result of this the shape and position of the front and rear sides of the lens basically change (accommodation). In the case of an intact accommodation system in a youthful person, the dioptric power of the system can vary by 14 dpt (breadth of accommodation) between the distance setting (disaccommodated state) and close-up setting (accommodated state). As a result, a youthful person with normal vision (emmetropia) is able to image focused objects on the retina, the objects lying between a far point at infinity and a near point approximately 7 cm in front of the cornea.

Since the ability of the human eye to accommodate reduces with increasing age, a number of artificially implantable lens systems with a variable focus have been developed.

Potentially accommodative intraocular lenses are lenses or lens systems which are inserted in place of the natural lens after the latter has been surgically removed and which are predominantly attached in the capsular bag. Haptics is intended to be applied to axially displace the lens by using a weak residual contraction of the ciliary muscle which is still available.

By way of example, DE 94 22 429 U1, DE 201 11 390 U1, DE 100 62 218 A1, DE 101 39 027, WO 02/083033, DE 101 25 829 A1, US 2004/0181279A1, US 2002/0149743, U.S. Pat. No. 6,120,538, U.S. Pat. No. 6,120,538, DE 101 55 345 C2, U.S. Pat. No. 6,096,078, U.S. Pat. No. 6,638,304, U.S. Pat. No. 6,638,304, WO 00/4605 and WO 00/4605, inter alia, all disclose a multiplicity of the developments.

Furthermore, DE 101 55 345 C2, U.S. Pat. No. 6,638,304 B2, WO 03/017873 A1 and U.S. Pat. No. 4,373,218 disclose apparatuses for restoring the accommodative capacity.

Furthermore, there are a number of scientific publications relating to the accommodative capacity of lens systems.

Reference is made in an exemplary manner to the following publications:

Schneider, H.; Stachs, O.; Guthoff, R.: Evidenzbasierte Betrachtungen zu akkommodativen Kunstlinsen [Evidence-based observations on accommodative artificial lenses], 102. Jahrestagung der Deutschen Opthalmologischen Gesellschaft [102nd Annual convention of the German Opthalmological Society] (Berlin, Germany, Sep. 23-26, 2004) (2004); Kammann, J.; Dornbach, G.: Empirical results refarding accommodative lenses. In: Current Aspects of Human Accomodation. Eds.: Guthoff, R.; Ludwig, K. Kaden Verlag Heidelberg (2001) 163-170, Fine, H.; Packer M.; Hoffmann R.: Technology generates IOL with amplitude of accommodation (Opthalmology Times Special Report, Mar. 15, 2005) (2005), Lavin, M.: Multifocal intraocular lenses—part 1. Optometry Today 5/2001 (2001) 34-37; Lavin, M.: Multifocal intraocular lenses—part 2. Optometry Today 8/2001 (2001) 43-44; Nishi, O.; Nishi, K.; Mano, C.; Ichihara, M.; Honda, T.: Controlling the capsular shape in lens refilling. Archives of Opthalmology 115(4) (1997) 507-510; Fine, I. H.: The SmartLens—a fabulous new IOL technology. Eye World 7(10) (2002).

The problem of accommodation to a reading distance of approximately 30 cm is not yet solved satisfactorily by the previous prior art. That is to say, in principle, the artificial lens implanted during a cataract extraction is unable to focus satisfactorily to different distances. Biological reasons mean that previous attempts of utilizing intraocular structures, in particular the ciliary muscle activity, to mechanically change the refraction of implantable systems have up until now been unsuccessful and nor is this to be expected in the medium term.

The prior art discloses solutions for intracoporeal determination of the accommodation requirement using the eyeball orientation of the pair of eyes. However, these are limited to detecting the contraction of the outer eye muscles, either by measuring the potential (e.g. U.S. Pat. No. 6,638,304) or by measuring the pressure difference between the eyeball and two horizontal bulbus muscles on different eyes (WO 2004/004605A1).

Generating strong, alternating electromagnetic fields and detecting the motion of small coils assumes equipment which is not suitable for implantation due to its mass and volume. Measuring the contraction of the bulbus muscles via the muscle potential or the pressure assumes a data connection between the sensor and the optically active implant in the capsular bag. A wired connection would greatly increase the surgical complexity. Wireless data transmission assumes an active system on the muscle, which would also require an energy supply. Additionally, using electrodes poses the problem of possible tissue changes in the region of the electrodes. It is for this reason that both solutions are not viable.

In order to determine the eyeball orientation in an implant, use has not yet been made of an external magnetic field. This is because previous solutions to measure the eyeball orientation using magnetic fields consist of coils in contact lenses, the spatial alignment of which can be determined by the interaction with strong, time-varying magnetic fields.

It is the object of the present invention to propose a system that can be implanted into the capsular bag and obtains its control impulses independently of the activity of the ciliary body.

This object is achieved by an implantable system for determining the accommodation requirement in an artificial accommodation system by measuring the eyeball orientation of both eyes using an external magnetic field, comprising
a) at least one optical system,
b) at least one data acquisition system which does not contact the ciliary muscle and has means for measuring a spatial orientation of both eyeballs as a physical control signal for the accommodation requirement,
c) at least one data processing system for generating an actuating signal for the optical system from the measured physical control signals,
d) at least one energy supply system, and
e) at least one fixing system, in which
   the system in each case has means for measuring a magnetic field in both eyes and provision is made for transfer means for mutual information exchange between the means.

The individual subsystems a) to e) of such an integral artificial accommodation system are described in the German patent application 102005038542 which was not published before the priority date of the current application. These systems are connected to form one or more control circuits. The optical system, the data acquisition system, the data processing system, the energy supply system and the fixing system are preferably combined to form an implant which can be inserted to restore the accommodative capacity of the animal or human eye using the fixing system. Here, the optical system is arranged in the beam path of the eye and, in conjunction therewith, forms the dioptric apparatus of the eye. In a similar fashion, the data acquisition system, the data processing system and the energy supply system are preferably arranged outside of the beam path. The data acquisition system can be distributed across a number of implants (e.g. in the left and right eyeball and in the upper jaw). The energy supply system can be connected, preferably wirelessly, to an external system.

The optical system, which comprises one or more active-optical elements and/or one or more rigid lenses which can be displaced axially by actuators (=passive-optical element), is intended to influence the imaging behavior in the beam path. It has to be transparent in the visible wavelength range and must be able to change, over time, the position and/or the shape of at least one of its refractive interfaces in order to change the dioptric power of the dioptric apparatus. The actuating component in this case comprises energy actuators and energy transducers (Grote/Feldhusen (Eds.): Dubbel—Taschenbuch für den Maschinenbau. 21. Auflage (Dubbel—Handbook for engineering. 21st editionl. Springer Verlag Berlin Heidelberg New York (2005)); when actuating signals of a data processing unit act on said actuating components, forces are put into effect which can then be converted into motion.

In the case of a passive-optical element, an actuator axially displaces one or more rigid lenses in the beam path. This active principle is routinely used in technical products for focusing. By way of example, DE4300840A1 describes a vario-objective for compact cameras comprising two lens groups whose mutual relative distance can be varied to effect a change in the focal length.

The above-described object of an active-optical element can be achieved using different mechanisms. In the process, it is necessary to distinguish between a change in the refractive index distribution and a change in the curvature of an interface separating two media with different refractive indices. These changes can be implemented by different physical action principles which are discussed below.

Change in the refractive index by electro-optical materials: Electromagnetic fields can influence the birefringent property of electro-optical materials. This makes it possible to set a defined refractive index distribution which affords the possibility of influencing the imaging behavior in a polarization plane of the light in a targeted fashion. In addition to a targeted change of the position of the focus, this can also comprise correcting higher-order image defects (e.g. astigmatisms, spherical aberration, coma). Two such systems have to be arranged in succession and cross at right angles in order to equally influence both mutually orthogonal polarization planes. U.S. Pat. No. 6,619,799 describes the use of such an active-optical element in a glasses frame. In the process, two transparent electrode surfaces enclose the electro-optical layer and an electrical voltage can be applied between said electrode surfaces in order to change the radial refractive index profile. A desired refractive index profile can be obtained by either modulating the amplitude and frequency of the control voltage or by dividing the electrodes into a number of regions which are respectively supplied with different voltages.

Change in the refractive index by changing the density of a compressible fluid: The refractive index of a compressible fluid (e.g. a gas or a gas mixture) depends on its density. This dependence is described by the Gladstone-Dale constant. If the pressure and/or the temperature are varied in a gas-filled chamber which has one or more curved interfaces, the imaging behavior of the optical system also changes accordingly. U.S. Pat. No. 4,732,458 describes, for example, such an arrangement for a multi-lens element whose refractive power can be changed continuously. The pressure increase in the rigid, gas-filled chamber is effected by a displaceable piston which is guided in a cylinder and arranged away from the optical axis.

Change in geometry as a result of an external force acting on an elastic solid body: An elastic solid body whose refractive index differs from that of the surroundings can be deformed by external forces so that the curvature of its light refractive surfaces changes and, as a result, this influences the optical imaging behavior. U.S. Pat. No. 6,493,151 describes, for example, an arrangement for a homogeneously or inhomogenously designed solid body which can be deformed in such a fashion and onto which radial forces can be transferred by means of a ring with a variable diameter. Thermal means or magnetic/electric fields can change the diameter of the ring. DE4345070 describes, for example, an arrangement for a deformable shell-shaped solid body which is filled by a transparent liquid and whose light refractive surfaces are hydraulically or pneumatically deformed by a ring-shaped fluid actuator. DE10244312 mentions the change in the refractive power of an artificial deformable lens implanted into the eyeball as an application example for an actuator composed of buckypaper (paper-like network of carbon nanotubes).

Change in geometry as a result of influencing the wetting angle (electrowetting): Two mutually immiscible fluids with approximately the same density but different refractive indices form a spherically curved or planar interface (meniscus). If the one, electrically conductive fluid is brought into contact with an electrode and a potential difference is applied with respect to a second electrode separated from both fluids by a dielectric layer, then the so-called electrowetting effect can change the wetting angle, and hence the curvature of the meniscus. Since the meniscus separates two media with differing refractive indices, there is a change in the optical imaging behavior. WO99/18456 describes an axial arrangement of conductive fluid, transparent dielectric and transparent electrode in the beam path and also measures for radially centering the tear in the optical axis. WO03/069380 describes an arrangement in which the dielectric-coated electrode is arranged cylindrically around the optical axis. The electrically conductive fluid and the insulating fluid, as well as the meniscus separating the two, are arranged axially one behind the other in the optical axis.

Change in geometry as a result of changing the pressure of a fluid: If the pressure difference in a fluid-filled chamber, having one or more deformable interfaces, and its surroundings is changed, this results in a change in the curvature of the interfaces and, accordingly, in a change in the imaging behavior of the optical system as well. U.S. Pat. No. 4,466,706 describes such an arrangement in an exemplary fashion, with a displacement mechanism changing the pressure difference. Here, turning a screw located in the cylindrical shell displaces fluid which leads to a change in the curvature of the two end faces of the cylinder. Alternatively, the shell can also be of a two-part design, with an axial relative movement of the two parts making such a displacement effect possible.

Change in geometry as a result of force developing within a smart material: Smart materials can develop forces by changing their atomic/molecular structure and, as a result of this, they can deform. The optical imaging behavior can likewise be influenced accordingly by setting an interface profile between the smart material and the surroundings. US2004/0100704 describes, for example, a shape memory polymer used for this purpose, which is inserted within a deformable lens body as a phase or layer and can locally change the shape of the body when influenced by energy. The post-operative, nonreversible correction of the imaging behavior of implanted intraocular lenses is mentioned as an exemplary application. JP01230004 describes using a swelling gel and a solvent arranged in layers within a deformable solid body in an exemplary fashion. The application of a voltage can effect a change in the solubility of the solvent in the swelling gel such that the latter thereupon undergoes a change in volume. This changes the curvature of the refractive surface.

Combinations of the abovementioned active principles are also possible. It follows that the optical system can adjust the focal position of the dioptric apparatus. Moreover, the optical system can comprise a plurality of elements in order to optimize the optical imaging behavior in the beam path. An active-optical element contained here may be able to correct (locally influence the light wavefront) further image defects (monochromatic and chromatic aberrations) in a static or dynamic fashion.

In order to generate actuating signals for the actuating component of the active-optical element or of the passive-optical element, it is necessary to acquire data from which the necessary dioptric power increase (=accommodation requirement) can be inferred.

The physical control signals from the eye movement can be used to obtain data about the accommodation requirements. Here, control signals are understood to be data containing the set-point value or the actual value, implemented under the influence of the set-point value, implemented under the signals, of a closed-loop control system. To be able to use the control signals from the eye movement, data from both eyes has to be used together to determine the required accommodation requirement.

In the case of binocular vision, the eye movement (in particular the horizontal vergence movement) and the accommodation requirement are clearly coupled to each other. The fixing lines of both eyes thus are aligned with a fixation object, arranged anywhere in space, by rotating the eyeballs so that the image of the fixation object falls on the corresponding retina locations. This makes it possible for the brain to fuse the data from the two individual images to a single image. The spatial orientation of the eyeballs can be described by the rotations of the eyeballs about the three spatial axes. Here, rotations about the horizontal axis (pitch movement), about the corotated vertical axis (yaw movements) and the corotated fixing line (roll movements) are considered separately in each eyeball. With reference to both eyes, it is accordingly possible to distinguish between conjugate eye movements (versions=movements of equal size and direction of the fixing lines or the retina meridians of both and disconjugate eye movements (vergences=movements of equal size but opposite direction of the fixing lines or the retina meridians of both eyes). In general, the distances between the fixation object and the two mechanical eye pivotal points, and hence the accommodation requirements, vary slightly (particularly when the fixation object is not symmetric with respect to the two eyes and close to the latter). The control signals of the eye movement (nerve signals or muscle signals) can be detected extracorporeally (e.g. by the electromyography of the eye muscles) but intracorporeally this measurement would be connected with high complexity. The motor of the eye movement is very precise, even in old age, and deviations between set-point and actual value (=fixation disparity) are only a few minutes of arc. It is for this reason that, as a very good approximation, a cut of the fixing line is ensured in binocular vision and it is possible to infer the accommodation requirement of the right or left eye from the effects of the nerve and muscle signals to the eye movement, i.e. from the orientation of both eyeballs in space.

It is possible to calculate the accommodation requirement from the position of the two eyeballs using the means for measuring the magnetic field. The fixation point is unambiguously fixed by the intersection of the fixing lines. The reciprocal of the distance between the cornea and the fixation point corresponds to the accommodation requirement. The accommodation requirement can basically be determined by the eyeball orientation, the angle subtended by the two fixing lines. If the fixation object lies on the perpendicular bisector of the two mid-points of the eyes, the accommodation requirement can be calculated exactly using the vergence angle. If the fixation object lies away from the perpendicular bisector, this calculation is sufficiently accurate.

By way of example, if the terrestrial magnetic field is used as a reference, the means for measuring the magnetic field can determine those angles subtended by the sensors, and hence the eyes as well, with respect to the magnetic field. The difference between the two angles corresponds to the vergence angle.

When a magnetic field is fixed with respect to the head, it is also possible to determine the version angle. Hence, in this case it is possible to determine the difference between the accommodation requirement of the left and right eye in the case of a fixation point away from the abovementioned perpendicular bisector.

According to the invention, provision is preferably made for two means for measuring a magnetic field which are fixed to the eyeballs. As a result of this, it is possible to measure the eyeball orientation using an external magnetic field, e.g. the terrestrial magnetic field or another magnetic field which, for example, can be fixed with respect to the head. By way of example, magnetoresistive or Hall sensors can be provided as means to measure a magnetic field. By way of example, they are designed using two mutually orthogonal sensors (compass sensor).

Hall sensors and magnetoresistive sensors, as well as magnetic field measurements, are generally known from the prior art.

Hall sensors utilize the Hall effect to measure magnetic fields and currents. If a current flows through a Hall sensor which is placed in a magnetic field running perpendicularly to said current, the Hall sensor generates a voltage which is proportional to the product of magnetic field strength and current. If the current is known, the magnetic field strength can be measured.

The magnetoresistive effect is based on the fact that magnetoresistance effects change the resistance of magnetic or nonmagnetic metals as a function of the direction (vector) and the magnitude of an external magnetic field.

Using the magnetoresistive or Hall sensors utilized according to the invention, it is possible to create, in pairs, an implant in each eye. The dimensions of the sensors are preferably in the range of 0.1 to 4 mm (edge length), with a thickness of 0.05 to 1 mm. That is to say, the preferred dimensions of the sensors are 5 mm×5 mm×2 mm, in particular 4 mm×4 mm×1 mm, and 1 mm×1 mm×0.05 mm is particularly preferred.

All sensors are preferably integrated into the system as chips without a housing. This is intended to avoid spatially intensive cabling in the overall system. Hence, this allows for the system to be applied on (e.g. in a contact lens) or in the eyeball.

Using an external magnetic field to measure the eyeball orientation allows for a positionally-independent measurement. This also affords the possibility of measuring in the implant in the capsular bag, and hence an integral implant can be obtained.

The possibility of an integral artificial accommodation system with only one implantation location, the capsular bag, significantly simplifies the implantation. Since the system is able to perform a measurement without electrical or tactile connection to the body, a sufficiently accurate measurement is possible, independently of possibly occurring tissue changes. This ensures a permanently functional system.

Since each implant independently measures its angular position using the magnetic field measurement, but the accommodation requirement has to be determined from the angles of both eyeballs, it is necessary to transfer data between the two implants. By way of example, alternating electromagnetic fields can be used to transfer this data between an implant in the left eye and one in the right eye. If alternating electromagnetic fields are used, it is possible to use the available magnetic field sensors as data receivers. This can be effected by alternately measuring and transferring, or else by selecting a carrier frequency which is selected to be that high that it cannot be influenced by interference signals caused by movements of the head or eyeball. In order to ensure that the system functions even in the case of temporally limited interferences of the terrestrial magnetic field, which do occur, it is possible to compensate the measurement errors generated by this by additional sensors which measure the angular acceleration in both eyes (gyroscope).

Within the scope of the invention described here, the data processing system is provided with the acquired data. However, the subject matter of the invention is also an above-described data acquisition system on its own, which can transmit measurement data to a receiver outside of the body for registering and further processing.

The acquired signals are processed by the data processing system (e.g. outlier tests, smoothing, filtering, amplifying). Features are extracted and classified using methods from classical statistics, computational intelligence and data mining in order to detect the accommodation intent. The required actuating signals for the optical system are generated using control and feedback control methods (e.g. fuzzy-controlled PID controller, adaptive control algorithms, learning algorithms). Both hierarchical control structures and central-decentral structures can be used.

An energy supply system, which may comprise an energy transducer, an energy storage device and a control unit, is used to supply the subsystems with energy. The energy transducer converts energy remotely transmitted from the outside (e.g. by inductive, capacitive or optical methods) or stored energy (e.g. battery, miniaturized fuel cell), which can also be available in the form of bodily fluids (e.g. the nutrient-rich aqueous humor, blood), or mechanical energy (e.g. from muscle movement) into electrical energy via an energy storage device. Said energy is emitted to the subsystems at precisely defined times by means of the control unit of the energy supply system. By measuring the strength of the illumination (e.g. by using a photocell), it is possible to bring the overall system into a state of minimal energy conversion in the case of darkness or closed eyes, i.e. in situations in which accommodative capacity is not required. The control signals required to this end are generated by the data processing system.

The overall system is implanted in the beam path using fixing elements which are suitable for axial fixing and radial centering. A number of haptical embodiments for intraocular lenses are known from opthalmology. (Draeger, J.; Guthoff, R. F.: Kunstlinsenimplantation [Artificial lens implantations]. In: Augenheilkunde in Klinik and Praxis Band 4 [In: Opthalmology in clinics and in practice Volume 4]. Eds.: Francois, J.; Hollwich, F. Georg Thieme Verlag Stuttgart New York (1991); Auffarth, G. U.; Apple, D. J.: Zur Entwicklungsgeschichte der Intraokularlinsen [On the development history of intraocular lenses]. Opthalmologe 98(11) (2001) 1017-1028). Said intraocular lenses can preferably be secured in the iridocorneal angle, in the ciliary sulcus or in the capsular bag.

The artificial accommodation system is the technical part of a control system (closed-loop control system) which, as an artificial system, replaces the function of the naturally deformable eye lens and the ciliary muscle of a patient. The biological part basically consists of: the cornea, the aqueous humor and the vitreous humor as components of the dioptric apparatus; the retina as a natural sensor array; and the brain as a natural data processing unit which generates control signals comprising data regarding the accommodation requirement.

The artificial accommodation system comprises an optical system with a variable focus and/or other optical properties. It forms a newly inserted component of the dioptric apparatus of the patient. It comprises a data acquisition system which has means to measure a magnetic field. A data processing system uses these measurements to determine the accommodation requirement and actuating signals for actuating the optical system are generated. The system is fed by a suitable energy supply system and is fixed in the patient's eye by means of a fixing system.

The described accommodation system can be used to restore the accommodative capacity after the natural eye lens has been removed due to a cataract or presbyopia.

In the following text, the invention is described in more detail with reference to the figures.

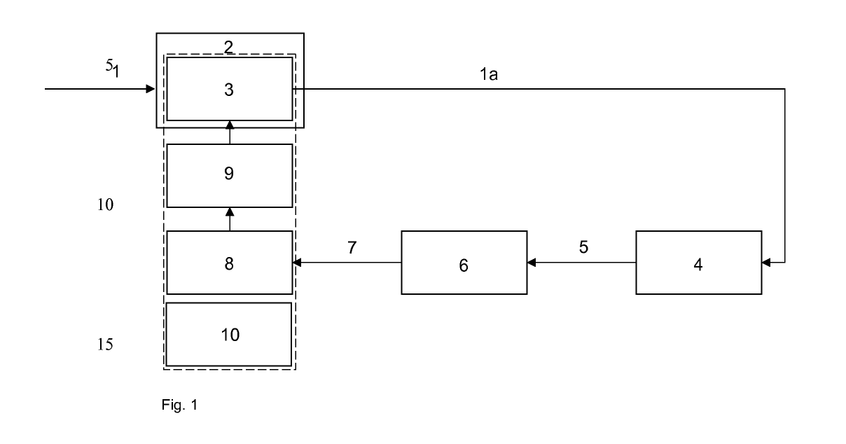

FIG. 1 reproduces a schematic illustration of the overall system (artificial accommodation system). The data 1, e.g. light from an object whose object distance varies over time, passes through the dioptric apparatus of the human eye 2, which comprises the optical system 3. The focused light 1a impinges on the natural sensor—the retina 4.

The afferent signals 5 generated by the photoreceptors are supplied to the natural data processing system 6—the brain. From there, efferent signals 7 comprising data regarding the accommodation requirement are sent to motor-driven structures (e.g. ciliary muscles, bulbus muscles). This data is picked up by the data acquisition system 8 of the artificial accommodation system. The data processing system 9 uses this to derive actuating signals for the optical system 3. Hence, the artificial accommodation system matches the dioptric power of the dioptric apparatus 2 to the accommodation requirement resulting from the temporally varying object distances. The energy supply system is represented by 10. All technical system components are framed by a dashed line.

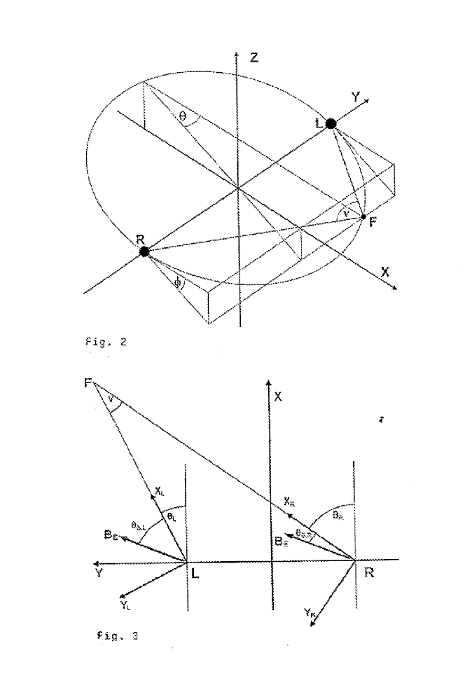

FIG. 2 illustrates the vergence angle ν, the version angle θ and the pitch angle Φ when observing a fixation angle F. Using this, the accommodation requirement can be calculated from the position of the two eyeballs. The fixation point of the fixation object F is prescribed by the intersection of the two fixing lines. The reciprocal of the distance between the cornea and the fixation object F corresponds to the accommodation requirement. The accommodation requirement can basically be determined by the vergence angle ν and the version angle θ. If the fixation object is on the bisector of the two eye centers, the accommodation requirement can be calculated exactly from the vergence angle ν.

FIG. 3 illustrates the position of the eyes with respect to the approximately homogeneous terrestrial magnetic field. The vergence angle ν can be measured using this terrestrial magnetic field by incorporating two compass sensors fixed to the eyeballs. By using the terrestrial magnetic field as a reference, the compass sensors make it possible to determine those angles which the sensors, and hence the eyes, subtend with respect to the magnetic field. The difference between these two angles corresponds to the vergence angle ν.

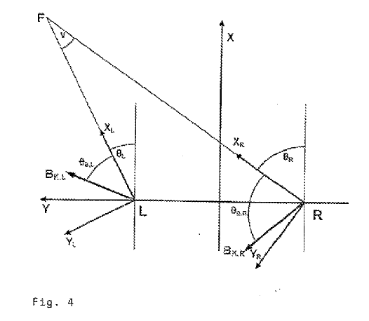

FIG. 4 illustrates the positioning of the eyes with respect to the arbitrary magnetic field which is, however, fixed with respect to the head. This is because it is possible to determine the version angle θ in addition to the vergence angle ν by using a magnetic field that is fixed with respect to the head. This in any case affords the possibility of determining the difference in the accommodation requirement between the left and the right eye if a fixation object F is away from the perpendicular bisector.

1. An implantable system for determining the accommodation requirement in an artificial accommodation system by measuring the eyeball orientation using an external magnetic field, comprising
a) at least one optical system (3),
b) at least one data acquisition system (8) which does not contact the ciliary muscle and has means for measuring a spatial orientation of both eyeballs as a physical control signal for the accommodation requirement,
c) at least one data processing system (9) for generating an actuating signal for the optical system (3) from the measured physical control signals,
d) at least one energy supply system (10), and
e) one fixing system,
wherein the system in each case has means for measuring a magnetic field in both eyes and provision is made for transfer means for mutual information exchange between the means.

2. The system as claimed in claim 1,
wherein the means for measuring a magnetic field are in each case fixedly connected to the respectively associated eyeball or are inserted therein.

3. The system as claimed in, claim 1, wherein the magnetic field is formed by a terrestrial magnetic field.

4. The system as claimed in claim 1, wherein the magnetic field is formed by a magnetic field that is fixed with respect to the head.

5. The system as claimed in claim 1, wherein the means for measuring the magnetic field respectively comprise a compass sensor or two magnetic field sensors which span a plane with their measurement directions.

6. The system as claimed in claim 5,
wherein the compass sensors or the planes in both eyes are aligned parallel with respect to a straight line, which connects the pivotal points of both eyes, and are mutually parallel.

7. The system as claimed in, claim 5, wherein the means for measuring the magnetic field are formed by magnetoresistive or Hall sensors.

8. The system as claimed in claim 1, wherein the measurement means can be used as transfer means and hence as data receivers for alternating fields and signals.
